(19) United States
(12) Patent Application Publication
(10) Pub. No.: US 2023/0025631 A1
(43) Pub. Date: Jan. 26, 2023

(54) NOVEL LACTOBACILLUS PLANTARUM STRAIN USEFUL FOR INTESTINAL CALCIUM ABSORPTION

(71) Applicant: VF BIOSCIENCE, Loos (FR)

(72) Inventors: Benoit CUDENNEC, Villeneuve dAscq (FR); François COUTTE, Villeneuve dAscq (FR); Pascal DHULSTER, Villeneuve dAscq (FR); Djamel DRIDER, Villeneuve dAscq (FR); Christophe FLAHAUT, Villeneuve dAscq (FR); Marc FREMONT, Loos (FR); Cyril RAVESCHOT, Loos (FR); Maxime VAEREMANS, Loos (FR); Shirchin DEMBEREL, Ulaanbaatar (MN); Jamyan DUGERSUREN, Ulaanbaatar (MN)

(21) Appl. No.: 17/772,638

(22) PCT Filed: Oct. 23, 2020
(86) PCT No.: PCT/FR2020/051926

§ 371 Date: Apr. 28, 2022

(30) Foreign Application Priority Data
Oct. 31, 2019 (FR) 1912308

(51) Int. Cl. A61K 35/747; C12N 1/20; A61K 31/593; A23C 9/123; A23C 11/10; A23C 9/127; A23C 19/06; A23L 33/135

(52) U.S. Cl. CPC A61K 35/747; C12N 1/205; A61K 31/593; A23C 9/123; A23C 11/106; A23C 9/127; A23C 19/062; A23L 33/135; C12R 2001/25

(57) ABSTRACT

The present invention relates to the isolated strain of Lactobacillus plantarum VF46A deposited at the CNCM (Collection Nationale De Cultures de Microorganismes, INSTITUT PASTEUR, 25 rue du Docteur Roux, F-75724 PARIS CEDEX15) under the number CNCM 1-5301 on 29 Mar. 2018 as well as to a pharmaceutical composition, a food supplement and a food comprising said strain.

The present invention relates to a novel probiotic strain able to modulate the intestinal absorption of calcium. More particularly, the present invention relates to a novel Lactobacillus plantarum strain.

As indicated in the publication entitled “Dietary and pharmacological compounds altering intestinal calcium absorption in humans and animals” of V. Areco et al, published in 2015 in the review Nutrition Research Reviews, volume 28, pages 83 to 99, calcium plays an important role in the maintaining of many biological functions such as bone metabolism, the cardiovascular system or the functioning of muscles and neurons. Pathologies such as osteoporosis, insulin resistance, obesity, arterial hypertension or metabolic syndrome, are associated with calcium deficiencies (or hypocalcemia).

In addition, the aforementioned publication indicates that a disturbed absorption of calcium can be at the origin of the occurrence of cancers such as colon, breast, prostate and ovarian cancer.

The majority of the calcium consumed is absorbed at the intestine, two calcium transport pathways have been identified in the intestine, the paracellular pathway and the transcellular pathway. The paracellular transport of calcium is a passive diffusion of this element from the lumen of the intestine (i.e. the internal space delimited by the wall of the intestine) to the intestinal mucosa, according to the gradient formed between these two compartments. Two proteins are involved in this transport, claudins 2 and 12 (CLD-2-12) which are part of the tight junctions between the cells and act as calcium channels. The mechanism is described in the publication entitled “Tight Junction Proteins Claudin-2 and -12 are Critical for Vitamin D-dependent Ca²⁺ Absorption between Enterocytes” of Fujita et al. published in 2008 in the review Molecular Biology of the Cell (volume 19, 1912-1921, May 2008).

The transcellular transport of calcium is an active transport involving the incorporation of calcium within intestinal cells by a transporter called TRPV6. The calcium is thereafter transported and excreted at the basal pole of the cell in the intestinal mucosa. These two calcium transport pathways are regulated by the vitamin D, a hormone that, once fixed to its nuclear receptor the VDR (Vitamin D Receptor) acts as a transcription factor controlling the genes of the CLD-2-12 and of TRPV6. The publication entitled “Mechanisms of Intestinal Absorption” of F. Branner published in 2003 in the review Journal of Cellular Biochemistry, page 387-393 and the publication entitled “The role of vitamin D in the endocrinology controlling calcium homeostasis” of J. C. Fleet, published in 2017 in the review Molecular and Cellular Endocrinology explain these mechanisms. By controlling the metabolism of the calcium, VDR acts on the bone remodeling and is involved in the pathologies that stem therefrom (osteopenia, osteoporosis, osteomalacia).

Furthermore, VDR is also involved in the defense of the organism against pathogens, in the detoxification of xenobiotics, in immunoregulation; it also exercises an anti-inflammatory and anti-cancer action and a cardiovascular protection (see the publication entitled “The nuclear vitamin D receptor controls the expression of genes encoding factors which feed the “Fountain of Youth” of M. R Haussier and published in the review Journal of Steroid Biochemistry and Molecular Biology in 2010). By regulating the intracellular calcium levels, the transporter TRPV6 itself will modulate processes such as proliferation and apoptosis which are controlled by signaling pathways involving calcium and play a crucial role in the occurrence and the development of cancers.

The results indicated in the publication entitled “Lack of Vitamin D Receptor Causes Dysbiosis and Changes the Functions of the Murine Intestinal Microbiome” of Jin et al, published in 2015, in the review Clinical Therapeutics, volume 37, issue 5, showed that a decrease in the activity of VDR results in a dysbiosis of the microbiota itself leading to profound alterations of the intestinal metabolism.

Lactobacilli constitute a safe and promising approach in the prevention of hypocalcemia. In this field, the state of the art shows that the impact of strains of Lactobacillus on the absorption of calcium has already been demonstrated by in vitro experiments. The action of a strain of L. salivarius on Caco-2 cells has already been studied. Furthermore, the impact of bacterial strains was mostly studied through in vivo studies where the beneficial action of bacteria is demonstrated at the skeleton and bones.

The publication entitled “Vitamin D receptor pathway is required for probiotic protection in colitis” of Wu and published in the revenue Am J Physiol Gastrointest Liver Physiol 309:G341-G349, in July 2015 shows that a strain of L. plantarum made it possible to increase the expression of VDR in murine and human intestinal cells. The strain also allowed for an increase in the expression of cathelicidin, an antimicrobial peptide. This same treatment in mice suffering from colitis made it possible to protect the intestine; through the VDR, the strain of L. plantarum results in an increase in the number of Paneth cells (immune cells of the intestine) as well as a decrease in the secretion of pro-inflammatory cytokines.

The aforementioned publication indicates that the bacteria can interact in diverse ways with the cells of their host. It also indicates that a strain of L. plantarum is capable of increasing the expression of the gene Cyp24, which is responsible for the transformation of the vitamin D present in the blood circulation, in the active form of this vitamin via the production of an enzyme called 24-hydroxylase. In other terms, this strain can potentially favor the fixation of blood calcium by the bone mass.

A purpose of the present invention is to propose a novel lactic bacterium strain capable of favoring the absorption of calcium, in an individual that can be a mammal, in particular a human.

Another purpose of the present invention is to propose a pharmaceutical composition containing such a strain and able to be used for the treatment of an abnormal physiological condition that can be a pathology or a syndrome, for example, and linked to the intestinal absorption of calcium and more particularly to an altered and excessively low absorption of the calcium by the intestine.

Another purpose of the present invention is to propose a food supplement and/or a food containing the strain of the invention.

The present invention relates to the isolated strain of Lactobacillus plantarum VF46A deposited on 29 Mar. 2018 at the CNCM under the number CNCM I-5301.

The Applicant has shown that surprisingly, this strain is able to increase the absorption of calcium at the intestine by favoring the passage of the calcium present in the intestinal lumen (the interior of the intestine) to the blood circulation through the epithelial cells of the intestine.

Indeed, although it was known from the aforementioned publication of Wu that a strain of L. plantarum was capable of increasing the fixation of the calcium present in the blood on the bones via the increase in the active form of vitamin D, it was not evident that such a type of strain can act directly on the intestinal absorption of the calcium.

The present invention also relates to the aforementioned strain for use as a drug.

Moreover, the strain of the invention also has an influence on the VDR since it activates the expression of the protein at the intestine. Potentially, the strain can therefore have a therapeutic effect on all pathologies that are linked directly or indirectly to a disruption involving VDR and/or proteins of which it regulates the expression, such as TRPV6, for example.

Thus, the strain of the invention can be used as an anti-inflammatory drug, an immunoregulator, for the prevention or the treatment of cancers, in particular of the colon, or for the treatment of diseases resulting from an alteration of the intestinal metabolism, such as for example CIBDs (chronic inflammatory bowel diseases).

Due to the effect of the strain of the invention on the absorption of calcium, it potentially has a therapeutic effect on all cardiac pathologies linked to the blood calcium level, in particular hypertension.

The term “treatment” encompasses in terms of the present invention the curative treatment and the preventive treatment.

The present invention also relates to the strain of the invention for use as a drug in the treatment of an abnormal physiological condition linked to the intestinal absorption of calcium, in particular linked to a deficit of the intestinal absorption of calcium and more particularly for the treatment of a pathology or of a syndrome selected from osteoporosis, type 2 diabetes, hypertension, cardiac pathologies linked to blood calcium, obesity, metabolic syndrome, cancer, in particular colon cancer, prostate cancer, ovarian cancer, breast cancer, pathologies linked to an alteration in the intestinal metabolism, in particular chronic inflammatory bowel diseases, for use as an immunoregulator or anti-inflammatory drug or for use in the treatment of an abnormal physiological condition linked directly or not to a malfunction of the vitamin D receptor.

The term “abnormal physiological condition” means in terms of the present invention, any physiological state defined by at least one parameter considered as indicative of a disorder or of a physiological disruption.

Thus, the abnormal physiological condition can be a pathology i.e. a set of symptoms of which the cause or the causes are identified or a syndrome i.e. one or more asymptomatic disruptions of which the cause or causes are not identified.

The present invention also relates to a pharmaceutical composition that contains as an active ingredient, the strain according to the invention and a pharmaceutically acceptable excipient.

This pharmaceutical composition can be used for the curative or preventive treatment or for the improvement of at least one of the symptoms of the same pathologies as those mentioned in reference to the strain of the invention.

The excipient can be a mixture of excipients. It can be selected from water, glycerol, ethanol, propylene glycol, vegetable oils, waxes, natural or synthetic sugars, glycerin and mixtures thereof, silica, talc and surfactants.

The pharmaceutical composition is formulated more preferably to be ingested; it is therefore preferably administered orally.

The pharmaceutical composition of the invention can have the form of a gel, liquid, suspension, tablet, lyophilizate or capsule.

By way of example, a unit dose of the pharmaceutical preparation of the invention contains 10⁵ CFU of the strain of the invention.

By way of a non-limited example, the daily dose in the strain according to the invention is at least 10⁵ CFU, preferably from 10⁹ CFU to 10¹⁰ CFU.

The present invention also relates to a food supplement containing the strain of the invention. The food supplement can have the same forms as those mentioned hereinabove in reference to the pharmaceutical composition of the invention. A unit dose of the food supplement of the invention can advantageously contain at least 10⁵ CFU of the strain according to the invention.

Advantageously, the food supplement contains at least 10⁵ CFU of said strain.

Advantageously, the food supplement contains calcium and/or vitamin D. The simultaneous presence of calcium and of the strain of the invention in the intestine favors the passage of the calcium to the blood circulation and the fixation thereof on the bones.

According to a particularly advantageous embodiment, the food supplement contains for a dosage unit 10⁹ CFU or 10¹⁰ CFU of the strain according to the invention.

The food supplement can be used as a supplement to a drug and for the treatment of the same pathologies as those mentioned in reference to the strain of the invention.

The present invention also relates to a food with the exception of yaourt obtained from dzomo milk, that contains the strain according to the present invention.

The food is advantageously selected from fermented foods.

The food of the invention can be selected, for example, from yogurts with the exception of dzomo milk yogurts, fresh or fermented cheeses obtained from milk of animal or plant origin, milks of animal or plant origin, kefirs of milk of animal or plant origin and preparations obtained by coagulation of casein.

The term food includes in terms of the invention beverages and more solid foods. The food according to the invention is advantageously selected from yogurts obtained from animal or plant milk with the exception of dzomo milk. The yogurt of the invention can be obtained from a mixture of milks of animal or plant origin. The food according to the invention can be a mixture of lacto-fermented vegetables of the sauerkraut type for example or of cured meats, in particular a sausage. Likewise, the type of milk from which the cheese according to the invention is obtained is not limited.

The foods obtained with an animal origin milk base have the advantage of also containing calcium which acts in synergy with the strain of the invention.

The food according to the invention can also be milk of animal or plant origin and containing the strain of the invention.

The food can, for example, contain 10³ to 10⁶ CFU of the strain according to the invention. In particular, it can contain 10⁵ CFU or more of the strain according to the invention.

The milk of animal origin can be selected from cow's milk, goat's milk, ewe's milk, mare's milk, donkey's milk and the mixtures of two or more of these milks.

The milk of plant origin can be selected from cereal milks such as oat milk, Kamut wheat milk, spelt milk, millet milk, barley milk, rye milk, rice milk, almond milk, soy milk, walnut milk, coconut milk, hemp milk, milk from oils such as hazelnut, peanut, cashew or pistachio, for example.

The present invention also relates to a composition selected from food or pharmaceutical compositions characterized in that it contains the strain according to claim 1 and the strain of Lactobacillus helveticus VFH049 deposited at the CNCM under order number CNCM-I-5403.

Such a composition has shown to be effective in the intestinal absorption of calcium due to the synergy of the two strains.

### BRIEF DESCRIPTION OF THE DRAWINGS

FIG. 1 shows the logarithm of the concentration of viable cells of the strain VF46A according to the stages of the gastrointestinal digestion simulated in vitro; the left-most rectangle corresponds to the concentration in the mouth, the central rectangle corresponds to the concentration in the stomach while the right-most rectangle corresponds to the concentration in the small intestine;

FIG. 2 shows the percentage of lactate dehydrogenase (LDH) released following the possible lysis of the cells (Caco-2 or HT-29 MTX) after a contact of 24 hours with the strain VF46A in relation to the control;

FIG. 3 shows the measurement of the transepithelial electrical resistance (TEER) of a monolayer of Caco-2 cells cultivated on insert either with the strain VF46A or with PBS, as a function of time;

FIG. 4 shows the ratio of the basal calcium concentration (basal concentration measured/basal concentration at t=0 h) as a function of time obtained with a monolayer of Caco-2 cells cultivated with PBS or the strain of the invention;

FIG. 5 shows the ratio of the fluorescence emission (EMIt/EMIt0-9 s) obtained as a function of time on a monolayer of Caco-2 cells cultivated with PBS (dotted line curves) or with the strain of the invention (solid line curves);

FIG. 6a shows the mRNA level of the VDR in relation to the control obtained for HT-29 MTX cells cultivated in the presence of PBS or in the presence of the strain of the invention; and

FIG. 6b shows the mRNA level of trpv6 in relation to the control obtained for HT-29 MTX cells cultivated in the presence of PBS or in the presence of the strain of the invention; and

FIG. 7 shows the level of cell proliferation within a culture of HT-29 MTX cells (cell line of cancer cells of the colon) (percentage of cell in G2/M phase) in a control medium (DMEM) or in a medium containing the strain VF46A.

### EXPERIMENTAL PART

### Isolation of the Strain

The strain of the invention was isolated from a yogurt obtained by fermentation of the milk of a dzomo in Mongolia and more particularly in the Mongolian region of Altai. A dzomo is a female resulting from the cross between a yak and a domestic cow. The aforementioned yogurt also contained other lactobacilli and in particular Lactobacillus delbrueckii subsp. bulgaricus and Streptococcus thermophilus.

The yogurt samples were collected in sterile containers and stored for a maximum of 2 days at −4° C. before analysis. The samples were successively diluted in a saline solution of NaCl at 0.85% (m/v) then spread on De Man, Rogosa and Sharpe (MRS) agar (De Man et al., 1960). After 2 days of incubation at 37° C. in anaerobic conditions, the morphologically distinct colonies were selected then spread again until purification. The strains were examined under the microscope after Gram staining. A search for catalase was conducted by adding hydrogen peroxide (H₂O₂) on the cultures.

The isolated strain is then stored in MRS medium supplemented with glycerol at a final concentration of 15% (v/v) at −80° C.

The identification of the strain was carried out by amplification and sequencing of the gene coding rDNA 16S. The DNA of the strains was extracted from colonies using the QIAamp DNA kit (Qiagen, Hilden, Germany).

The amplification of the DNA was carried out by the company GATC Biotech (Constance, Germany) using the primers 27F (5′-AGAGTTTGATCCTGGCTCAG-3′ (SEO ID NO 7) and 534R (5′-ATTACCGCGGCTGCTGG-3′ (SEO ID NO 8). This technique is described in the publication of Muyzer et al., dated 1993. The sequencing was carried out by this same company using the primer 27F. The bacterial species were identified by comparing the sequence of the gene 16S with the sequences of the Medline database, a score greater than 99% was considered to be significant.

The strain VF46A was identified as being a strain of Lactobacillus plantarum and was deposited at the CNCM (Collection Nationale de Cultures de Microorganismes INSTITUT PASTEUR 25 rue du Docteur Roux, F 75724 PARIS CEDEX 15) under the number CNCM I-5301 on 29 Mar. 2018.

The strain VFH049 was deposited at the CNCM (Collection Nationale de Cultures de Microorganismes INSTITUT PASTEUR 25 rue du Docteur Roux, F 75724 PARIS CEDEX 15) under order number CNCM-I-5403 on 19 Feb. 2019.

### Evaluation of The Characteristics of The Strain

### Tolerance to Acidity

The principle of this test is based on the comparison of the survival of a bacterial strain between an incubation of 2 h at pH 2 and an incubation at the pH of the MRS medium (pH 6.2). For this experiment, the strain was cultivated in a liquid MRS medium for 24 h at 37° C. in anaerobic conditions. A volume of 200 μL of culture was then diluted ( 1/10 ) either in the same MRS medium at pH 6.2 (called tube A), or in a MRS medium adjusted to pH 2 by hydrochloric acid (HCl) (called tube B). Tubes A and B were incubated at 37° C. for 2 h then 10 μL of each tube were spread on MRS agar. The plated dishes were incubated for 48 h at 37° C. in anaerobic conditions.

The tolerance to acidity was evaluated by comparing the number of colonies between dishes A and B. A scale of classes of tolerance to acidity was defined according to the result of the ratio B/A. Class 0 includes the strains that are not tolerant to acidity (no colony on dish B). Class 1 corresponds to the strains that are weakly tolerant (dish B=0 to 30% of the colonies on dish A). Class 2 includes the strains having a moderate tolerance to acidity (dish B=30 to 80% of the colonies on dish A). Finally, class 3 corresponds to the strains that are tolerant to acidity (dish B has more than 80% of the colonies of dish A).

The strain of the invention survived after a passage at pH 2 for 2 h. The results show that the strain VF46A is categorized in class 3 of tolerance to acidity i.e. more than 80% of the bacterial cells resisted the acid treatment.

### Parietal Hydrophobicity

The objective of this test is to evaluate the hydrophobic nature of the wall of the strain. A simple test has already been developed to evaluate this criterion, it is the MATS test (Microbial Adhesion To Solvents). It consists of measuring the affinity of a bacterial strain for an apolar hydrocarbons (n-hexadecane most often) in order to estimate the hydrophobicity of the wall, a parameter of the capacity of the strain to adhere to the intestinal epithelium.

For the experiment, the bacterial strain VF46A is cultivated in liquid MRS medium for 24 h at 37° C. in anaerobic conditions. The cells are then washed twice by successively repeating a step of centrifugation at 10,000 rpm for 10 min, followed by a step of resuspending cells in a Phosphate Buffered Saline (PBS) buffer pH 7.4.

The Optical Density (OD) at 630 nm (initial OD_{630 nm}) was determined with the ELx808 spectrophotometer (BioTek Instruments Inc., Vermont, United States). A volume of 1 mL of suspension was then mixed with 100 μL of n-hexadecane

(Acros Organics, Geel, Belgium) by vortexing the mixture for 1 min. The solution was then left to rest at room temperature for 15 min then 100 μL of the aqueous phase were recovered for another determination of the OD at 630 nm (final OD_{630 nm}) . The parietal hydrophobicity was calculated according to the following formula:

$$\% H = ( \frac{{OD}_{630 n m} initial - {OD}_{630 n m} final}{{OD}_{630 n m} initial} ) \times 100$$

With % H: the percentage of parietal hydrophobicity, initial OD_{630 nm}: the initial OD of the suspension and final OD_{630 nm}: the OD measured after adding and mixing of the suspension with the n-hexadecane.

By measuring the affinity of the strain VF46A for a non-polar hydrocarbon, the parietal hydrophobicity of the strain was able to be estimated. The results indicate a percentage of parietal hydrophobicity equal to 32% for this strain.

### Study of Probiotic Natures

For this part, different criteria were studied. These criteria include different characteristics defining the probiotics in particular the capacity of adhesion to the intestinal epithelium, the innocuousness of the strains as well as their tolerance to gastrointestinal digestion (GID).

### Self-Aggregation Test

This test makes it possible to evaluate the capacity of bacterial cells to aggregate together in a liquid medium. It is based on the comparison of the bacterial concentration above a suspension at time 0 with the bacterial concentration of the same suspension incubated for a given time without any agitation. The strains capable of a strop self-aggregation will aggregate together thus increasing their sedimentation speed.

The strains are cultivated in MRS for 24 h at 37° C. then washed and resuspended in a PBS buffer at an OD at 600 nm of 1. A volume of 4 mL of suspension is vortexed for 10 sec then incubated at room temperature without agitation. After 2.5 and 5 h of incubation, 100 μL of medium are carefully sampled at the surface of the suspension. The samples taken are then diluted ( 1/10) in PBS buffer for a measurement of the OD at 600 nm.

The percentage of self-aggregation (% A) is then calculated according to the relationship:

$$\% A = ( 1 - \frac{{A}_{t}}{{A}_{0}} ) \times 100$$

With A₀: the absorbance at initial 600 nm of the suspension, measured at the beginning of the experiment (and equal to 1) and Aₜ: the absorbance at 600 nm measured at 2.5 or 5 h of incubation.

Regarding the strain VF46A, an increase in the self-aggregation between 2.5 and 5 h is observed. Indeed after 5 h of incubation, the strain has a percentage of self-aggregation equal to 28.27% (compared to 12.74% at 2.5 h).

In parallel, the adhesion capacity of the strain VF46A (see hereinafter) was measured by using the Caco-2 cell line. The strain VF46A is particularly adherent to the intestinal cells with 4.63% (±1.22) of cells adherent after contact.

The self-aggregation and the adhesion capacity of the strain VF46A indicate that the latter has a very good colonization capacity of the intestinal epithelium.

### Evaluation of the Tolerance to Gastrointestinal Digestion

In order to evaluate the survival of the bacterial strain during and after its passage within the digestive tract, a static digestion model was used. This model simulates the first 3 compartments of the digestive tract namely the mouth, stomach and the intestine sequentially. Each compartment is simulated by the adding of a fluid mimicking the physiological conditions of this step. All the digestion is carried out in sterile conditions, all the fluids are autoclaved at 121° C. for 20 min before the experiment, the adding of enzymes takes place after sterilization and is followed by a sterilizing filtration (at 0.2 μm).

The strain is cultivated in a MRS medium for 24 h at 37° C. The bacterial cells are washed twice then resuspended in a PBS buffer at a concentration of 10⁹ Colony Forming Units (CFU)·mL:⁻¹. For the digestion, the buccal phase is simulated by the adding of 8 mL of PBS, adjusted to pH 6.8, to 1 mL of the bacterial suspension. The mixture is incubated under a constant agitation at 200 rpm for 5 min at 37° C. The gastric phase is simulated by the adding of 12 mL of PBS at pH 3 supplemented with bovine pepsin (Sigma-Aldrich, St Louis, United States) at 1.56 mg·mL⁻¹. The suspension is incubated 2 h at 37° C. with agitation at 200 rpm. During the incubation, the pH of the solution is maintained between 3 and 3.5 by the adding of hydrochloric acid (HCl) (1 M) or sodium hydroxide (NaOH) (1 M). For the intestinal phase, 1 mL of sodium carbonate (NaHCO₃) at 1 M is added to the mixture so as to increase the pH to about 7 and to inactivate the pepsin. A volume of 12 ml of PBS at pH 8.2 supplemented with bovine pancreatic enzymes (Sigma-Aldrich, St Louis, United States) at 0.28 mg·mL⁻¹, then a volume of 6 mL of PBS at pH 8.1 containing 60 g·L⁻¹ of Ox-bile (Sigma-Aldrich, St Louis, United States) are added to the mixture so as to simulate the intestinal conditions. This step unfolds for 2 h at 37° C. with agitation at 200 rpm by maintaining the pH between 7 and 7.5.

At the end of each step of the digestion, a sample of 100 μL of the reaction mixture is taken, thus for the same digestion, 4 samples are obtained comprising the sample from the guide tube (TM), of the salivary phase (S), of the gastric phase after 2 h of incubation (G2), and finally the one after 2 h of intestinal phase (I2). Each sample is then diluted ( 1/10) successively in a PBS buffer. For the determination of the bacterial concentration in the samples, 100 μL of the adequate dilutions are spread on an MRS agar. The plated plates are then incubated at 37° C. for 48 h in anaerobic conditions. After incubation, the enumeration of the CFUs allows for the determination of the bacterial concentration in the samples. The results are expressed in CFU·mL⁻¹.

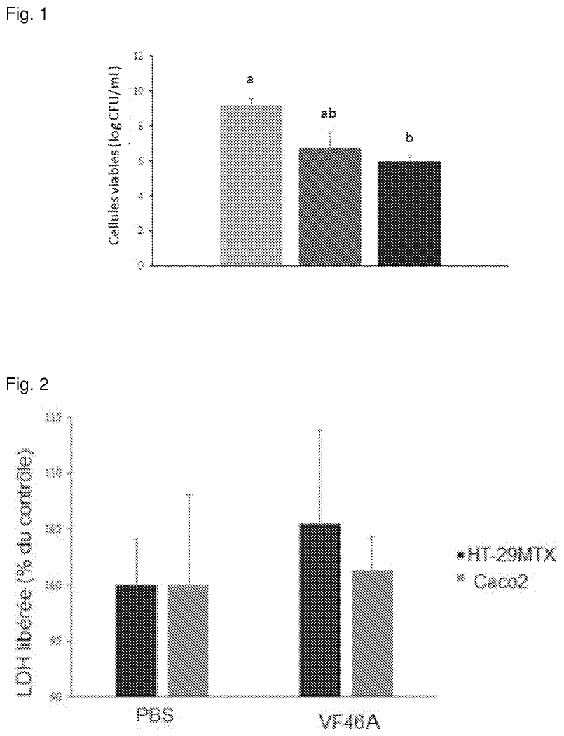

The results obtained for the strain VF46A are shown in FIG. 1. The initial number of viable bacterial cells is 10⁹ CFU·mL⁻¹ in the salivary phase (the left-most rectangle). During the GID, the quantity of bacteria decreases, the gastric phase being the most deleterious (central rectangle). At the end of GID (the right-most rectangle), the number of viable bacterial cells loses 3 log points in comparison with the number initially added at the beginning (10⁶ CFU·mL⁻¹ at the end of GID).

However, the aforementioned results indicate that the strain supports the digestion and can therefore be found unaltered in the small intestine of a subject.

### Cytotoxicity Test

The objective of this test is to evaluate the possible toxicity of the strain with regards to the intestinal barrier. Caco-2 and HT-29 MTX cells are used for this purpose.

For the cytotoxicity test, the cells are plated in a 96-well plate at a density of 8,000 cells per well in a volume of 150 μL of medium and cultivated for 7 days at 37° C., 5% CO₂. Before adding bacterial strains, the intestinal cells are washed twice with PBS buffer. The bacterial strains are cultivated as described hereinabove and suspended in the DMEM medium without addition at a concentration of 10⁷ CFU·mL⁻¹. For each well, 150 μL of bacterial suspension are added. PBS buffer, diluted with DMEM without addition as for the strain, is used as a negative control. The contact between the strain and the intestinal cells takes place for 24 h at 37° C., 5% CO₂. The strain is put into contact both with the Caco-2 cells and the HT-29 MTX cells independently.

The determination of the mortality of the intestinal cells is used here to evaluate the possible toxicity of the bacterial strains. The mortality of the cells is evaluated by dosage of the activity of the lactate dehydrogenase (LDH). This activity is dosed by the LDH activity assay kit (Sigma-Aldrich, St Louis, United States), this kit is based on the reduction reaction of oxidized nicotinamide adenine dinucleotide (NAD) in its reduced form (NADH) by the lactate dehydrogenase, with NADH able to be detected at 450 nm.

After contact, 50 μL of supernatant are sampled in each well and the activity of the LDH is dosed according to the protocol of the kit. The absorbance of the plate is read at 450 nm by a Xenius XC spectrofluorometer (Safas Monaco, Monaco, France). The mortality of the intestinal cells is calculated as a percentage of the average of the absorbances of the control condition (the proportion of LDH released in this condition is taken for reference at 100%).

The PBS buffer was used as a reference (control) to represent the basal level of released LDH. The results show an absence of significant toxicity to the two cell lines, which suggests that in these experimental conditions, the bacterial strain of the invention is not deleterious for the intestinal cells.

### Evaluation of the Adhesion of the Strains to the Intestinal Cells

The objective of this test is to evaluate the capacity of the strain to adhere to intestinal cells. It is based on the comparison between the quantity bacterial cells adhering to the monolayer of Caco-2 cells in relation to the quantity of bacteria initially added.

The strain is cultivated and prepared such as described hereinabove, a suspension in a DMEM medium without addition is prepared at a concentration of 10⁷ CFU·mL⁻¹. The Caco-2 cells are plated in 24-well plates at a concentration of 40,000 cells per well in a volume of 500 μL of complete DMEM. After incubation for 7 days at 37° C., 5% CO₂, the cells are washed twice with a PBS buffer.

A volume of 300 μL of bacterial suspension is added to each well, the plate is then incubated at 37° C., 5% CO₂, for 2 h. The bacterial suspension used is stored for determination of the concentration in CFUs. After 2 h of contact, the Caco-2 cells are washed twice with PBS buffer in order to remove the non-adhering bacteria. The intestinal cells are then lysed by the adding of 100 μL of PBS buffer supplemented with Triton X-100 at 0.1% (v/v). After incubation for 15 min at room temperature, the lysate and the initial suspension used are diluted successively ( 1/10) in PBS, then spread on an MRS agar. The plated plates are incubated 48 h at 37° C. in anaerobic conditions. After enumeration and determination of the bacterial concentration in CFU·mL⁻¹, the percentage of adhering bacterial cells is calculated in relation to the concentration obtained in the initial suspension representing the quantity of bacteria added at the beginning of the experiment (fixed at 100%).

As indicated hereinabove, the strain VF46A is particularly adherent to the intestinal cells with 4.63% of cells adhering after contact.

### Study of the Impact of the Strains on the Metabolism of Calcium and of Vitamin D

### General Contact Protocol Between the Bacterial Strains and the Intestinal Cells

This contact protocol is used for each technique used in this part. The bacterial strain is cultivated in MRS for 24 h at 37° C. in anaerobic conditions. The cells are recovered by centrifugation at 10,000 rpm for 10 min then resuspended in a PBS buffer, this step is repeated a second time to wash the bacterial cells. A bacterial suspension is finally prepared with DMEM medium without addition at a concentration of 10⁷ CFU·mL⁻¹.

The HT-29 MTX cells are plated in a 24-well plate at a concentration of 40,000 cells per well in a volume of 500 μL of complete DMEM. The Caco-2 cells are plated in a 96-well plate at a concentration of 8,000 cells per well in a volume of 200 μL of complete DMEM medium. For another experiment, the Caco-2 cells are plated on inserts placed in 12-well plates (polyester membrane, 0.4 μm, Costar®, Corning, N.Y., United States) apical side in a volume of 500 μL of complete DMEM medium, a volume of 1.500 μL of this same medium is added on the basal side of the insert. All the cell cultures are cultivated for 15 days with a renewal of the medium every 2 days during the second week of culture.

The intestinal cells are washed twice with the PBS buffer before the contact with the bacterial strain. A volume of 150 μL of bacterial suspension is added in each well for a culture in a 96-well plate, and a volume of 300 μL is added for a contact in a 24-well plate or with inserts on a 12-well plate. In any case, the contact takes place for 24 h at 37° C., 5% CO₂. The negative control used is a PBS buffer diluted in complete DMEM medium.

### Measurement of The Total Transport of Calcium

The total transport of calcium is defined as being the portion of calcium passing from the apical pole (which corresponds to the interior of the intestine) to the basal pole (which corresponds to the blood circulation) passing through the epithelial barrier. This transport is evaluated by the variation in the concentration in calcium at the basal pole of the cell barrier over time. The Caco-2 cells cultivated in inserts in a 12-well plate are used for this experiment.

At the beginning of the contact with the bacterial strain (t=0 h), 10 μL of a solution of calcium chloride (CaCl₂) at 250 mM are added on the apical side of the insert. During the contact, samples of 100 μL are taken from the basal side of the insert at 30 min, 7 and 24 h of contact. The samples are stored at −20° C. until the analysis.

At the time of the different samples, a measurement of the transepithelial electrical resistance (TEER) is taken to verify the integrity of the barrier of Caco-2 cells. This resistance is measured by a MilliCell Electrical Resistance System voltmeter/ohmmeter (Merck Millipore, Burlington, United States). An empty insert without cells is used as a blank to obtain the resistance of the cell barrier. The resistance (in Ω) of each well measured is then multiplied by the area of the insert (equal to 1.12 cm² for the insert used) in order to give a resistance in Ω·cm⁻². The results are expressed as a percentage of the value obtained at 30 min of contact (fixed at 100%) for each well.

In order to measure the total transport of the calcium, a dosage of the concentration in calcium is carried out in the samples taken at the basal pole of the membrane of Caco-2 cells during contact. The determination of the concentration in calcium is carried out using the calcium colorimetric assay kit (Sigma-Aldrich, St Louis, United States). This kit is based on the calorimetric reaction between the calcium ions and the orthocresolphtaleine forming a colored complex able to be detected at 575 nm (Morin, 1974). The dosage is carried out from 25 μL of samples diluted (½) in H₂O O Milli-Q®, following the protocol of the kit. The measurement of the absorbance of the samples at 575 nm is carried out by a Xenius XC spectrofluorometer (Safas Monaco, Monaco, France). The concentration in calcium in the samples is determined using a standard range of CaCl₂, it is then expressed according to the ratio of the concentration at time t over the concentration at time t=30 min of contact.

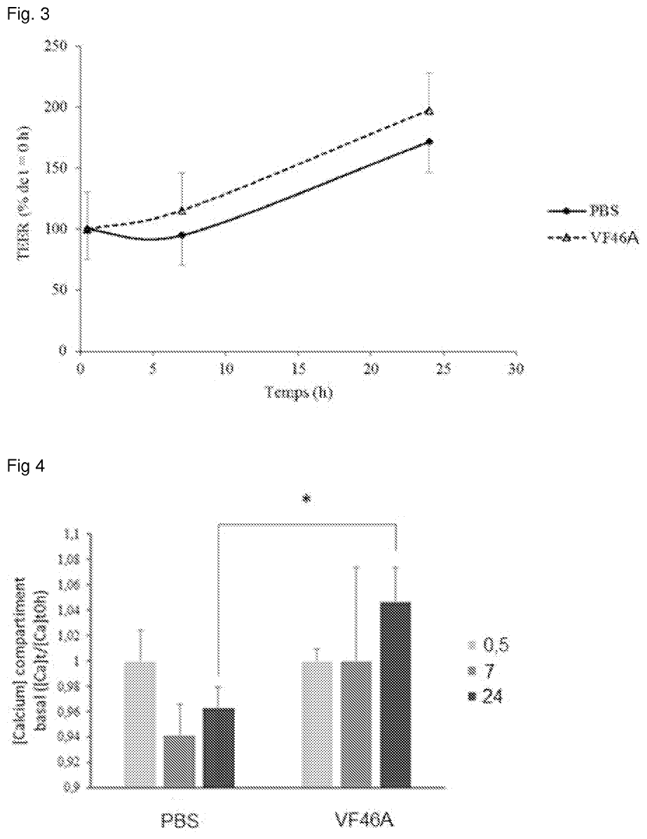

The results shown in FIG. 3 show that the TEER increases during the incubation time with the strain as well as in the control condition (PBS buffer). However, no significant difference was observed between an incubation with the bacterial strain and the PBS buffer which suggests that the strain VF46A does not cause any disturbances in the integrity of the membrane of Caco-2 cells during the experiment.

Regarding the total transport of calcium, the results of FIG. 4 indicate that the strain VF46A does not make it possible to significantly modulate the absorption of the calcium after 7 h of contact in relation to the PBS buffer. However, after 24 h of incubation, the concentration in calcium decreases in the basal compartment for the control condition, while a significant increase in the quantity of calcium is observed for the strain VF46A. These results suggest that the strain is capable of positively modulating the transport total of calcium from the interior of the intestine to the blood circulation through the intestinal wall, after 24 h of incubation with the Caco-2 cells.

### Measurement of the Incorporation of Calcium Within Intestinal Cells

Contrary to the preceding experiment, the objective here is to determine the capacity of the cells to incorporate calcium in the intracellular compartment. The incorporated calcium is detected by spectrofluorimetry using a fluorescent probe present in the cytoplasm of the cells. The Caco-2 cells plates in 96-well plates are used for this experiment, after contact with the bacterial strain, the cells are washed twice with a PBS buffer before incorporating the fluorescent probe.

The molecule used to detect the calcium is a Green FluoForte® probe provided with the FluoForte® calcium assay kit (Enzo Life Sciences, Farmingdale, United States). This probe is added on the cells in a non-fluorescent esterified acetoxymethyl (AM) form, once incorporated, the lipophilic group blocking the fluorescence is cleaved by non-specific cellular esterases, this results in the activation of the probe which will emit a fluorescence once bonded to the calcium. In its active form the probe cannot be rejected by the cellules due to the presence of an efflux inhibitor (added at the same time).

After rinsing Caco-2 cells, the probe is added by following the instructions of the kit, the plate is then incubated at room temperature for 1 h the penetration time of the probe. The fluorescence emission is then followed by a Xenius XC spectrofluorometer (Safas Monaco, Monaco, France) with an excitation wavelength at 490 nm for an emission followed at 525 nm. The emission is measured for 30 sec with an injection of 25 μL of a solution of CaCl₂ at 250 mM between 9 and 10 sec of kinetics by means of a module of injectors coupled to the spectrofluorometer. This injection causes a sudden entry of calcium within the intestinal cells leading to an increase in the fluorescence emission. The capacity of the strain to modulate the incorporation of the calcium is determined by this increase in fluorescence after injection of the calcium. The results are thus expressed for each well according to the average fluorescence measured between 0 and 9 sec of kinetics which is considered as the basal fluorescence of a well. A fluorescence increase ratio, for the different contact conditions, is therefore obtained.

The results visible in FIG. 5 indicate that an incubation with a PBS buffer (dotted line curves) results in an increase in the fluorescence ratio from 1 to 1.3-1.4. In comparison the strain VF46A (solid line) leads to a significant increase in the incorporation of calcium in relation to the control, the fluorescence ratio passing indeed from 1 to 1.4-1.5 suggesting a modulation capacity of the incorporation of calcium by the intestinal cells.

### Study of the Expression of Different Genes Involved in the Metabolism of Calcium and of Vitamin D

In this part, the changes in the expression of several genes after contact for 24 h between the strain and the HT-29 MTX cells were studied. The contact took place in a 24-well plate as described hereinabove.

After contact, the HT-29 MTX cells are rinsed twice with PBS buffer. The extraction of the RNAs is then carried out by the reagent TRIzol™ (Sigma-Aldrich, St Louis, United States).

The samples of RNA were first treated with DNase to remove any fragments of DNA co-extracted and/or remaining following the extraction. A volume of 8 μL containing 1.000 ng of RNA is mixed with 1 μL of DNase, and 1 μL of DNase buffer (ThermoScientific, Waltham, United States). The reaction takes place at 37° C. for 30 min in a thermocycler Mastercycler gradient (Eppendorf, Hamburg, Germany), it is stopped by the adding of 1 μL of EDTA solution at 50 mM (ThermoScientific, Waltham, United States) followed by an incubation at 65° C. for 10 min. Thereafter, the samples were retro-transcribed as cDNA using the RevertAid H minus first strand cDNA synthesis kit (ThermoScientific, Waltham, United States), according to the instructions provided.

For the qPCR reaction, the samples of cDNA are diluted ( 1/16) in H₂O. For a volume of 2 μL of sample, 18 μL of qPCR mixture are added containing 10 μL of Power SYBR® Green PCR Master Mix (2×) (Applied Biosystems, Life Technologies, Foster City, United States), 0.6 μL of each primer (10 μM) and 6.8 μL of H₂O. The fluorescence is monitored during the reaction in a thermocycler CFX Connect Real Time Detection System (Bio-Rad, Hercules, United States). After a step of denaturation at 95° C. for 10 min, 40 PCR cycles are carried out successively, one cycle comprises a denaturation at 95° C. for 15 sec, a hybridization from 58° C. to 61° C. according to the pair of primers used for 30 sec and an elongation at 72° C. for 30 sec. The production of a melting curve terminates the reaction.

In this part, 2 genes are studied, these are the gene coding for the vitamin D receptor (vdr) and finally the gene coding for the transporter of the calcium (transient receptor potential selective for calcium) (trpv6) also called CaT1 (calcium transporter 1). The expression of these genes is normalized in relation to that of the gene coding for peptidylprolyl isomerase A (ppiA) taken for reference. The pairs of primers used for the genes studied, as well as their hybridization temperatures, are presented in table 1 hereinbelow:

TABLE 1

| | | Size of the Tm | |
|---|---|---|---|
| Gene | Primers (5′-3′) | amplicon (bp) | (° C.) |
| vdr | Forward | : | |
| | GCCACCTGCTCTATGCCAAG | | |
| | (SEQ ID NO 1) | 171 | 51 |
| | Reverse | : | |
| | CAGGCTGTCCTAGTCAGGAGAT | | |
| | (SEQ ID NO 2) | | |
| trpv6 | Forward | : | |
| | TGATGCGGCTCATCAGTGCCAGC | | |
| | (SEQ ID NO 3) | | |
| | Reverse | 251 | 58 |
| | GTAGAAGTGGCCTAGCTCCTCG | : | |
| | (SEQ ID NO 4) | | |
| ppia | Forward | : | |
| | TGCTGACTGTGGACAACTCG | | |
| | (SEQ ID NO 5) | | |
| | Reverse | 136 | 30 |
| | TGCAGCGAGAGCACAAAGAT | : | |
| | (SEQ ID NO 6) | | |

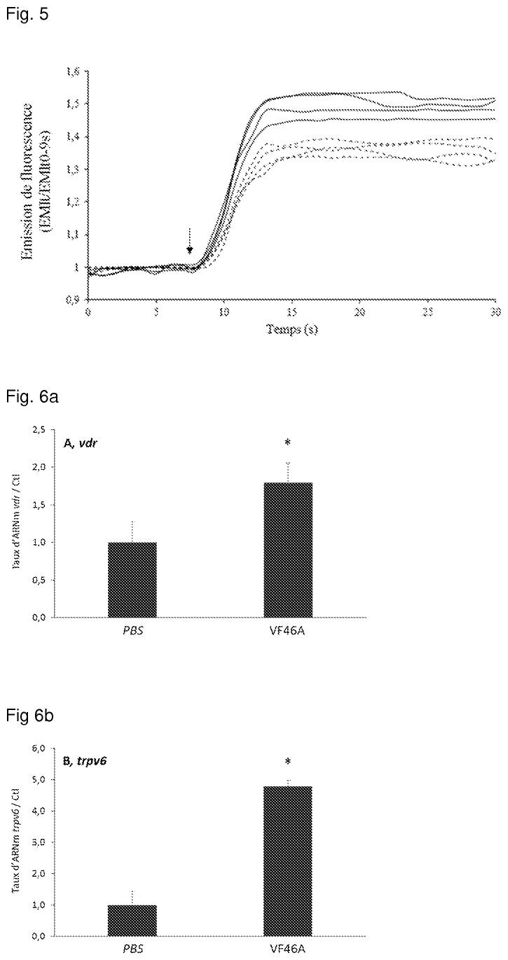

The results visible in FIGS. 6a and 6b show that the expression of the gene coding for the vitamin D receptor (vdr) is significantly increased in the presence of the strain VF46A. It is increased 1.8 times in relation to the expression in the presence of a PBS buffer (FIG. 6a). In addition, the treatment by the strain of the invention results in an increase in the expression of the gene coding for the transporter of the calcium (trpv6). The expression of this gene in the presence of the strain of the invention is at least 3 times greater than the control (FIG. 6b). In summary, the strain VF46A modulates the expression of the genes trpv6 and vdr in the HT-29 MTX cells. In light of the preceding results, the strain also made it possible to improve the incorporation of calcium by the Caco-2 cells. All of this data thus suggests a capacity of the strain VF46A to modulate the absorption of the intestinal calcium by acting on the transcellular pathways of the absorption.

### Test of Cell Proliferation on Cancer Cells

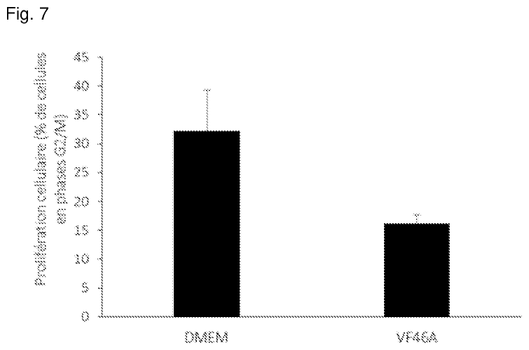

The strain of the invention is cultivated and prepared as described hereinabove; a suspension in DMEM medium is prepared at a concentration of 10⁷ CFU·mL⁻¹. The HT-29 MTX cells are plated in 24-well plates at a concentration of 40,000 cells per well in a volume of 500 μL of DMEM. After incubation for 11 days at 37° C., 5% CO₂, the cells are washed twice with a PBS buffer then incubated for 24 h in a DMEM medium. After incubation, a volume of 500 μL of bacterial suspension is added into each well and the plate is again incubated 24 h at 37° C., 5% CO₂. The control condition corresponds to an incubation in a DMEM medium without bacteria. After contact with the bacterial cells of the strain of the invention, the HT-29 MTX cells are washed twice with a PBS buffer then recovered by the adding of 200 μL of a trypsin/EDTA mixture. After centrifugation at 1,500 rpm for 5 min, 3 mL of DMEM medium are added so as to inactivate the trypsin then the cells are washed in a PBS buffer by repeating the centrifugation and resuspension steps. The cells are finally fixed in an ethanol solution at 66% for 12 h at 4° C. The evaluation of the cell proliferation was evaluated via the FxCycle™ PI/RNase Staining Solution kit (Thermo Fisher Scientific, Waltham, United States) followed by an analysis with a flow cytometer (Attune NxT Flow Cytometer, Thermo Fisher Scientific) making it possible to differentiate the cells in G0/G1 f phases from the cells in G2/M phases. The results are expressed as a percentage of cells in G2/M phase in relation to total number of cells analyzed. The results are visible in FIG. 7. In light of the results shown in FIG. 7, it is observed that the cancer cells that were put into contact with the strain of the invention divide less than the cancer cells that were not put into contact with the cells of the strain of the invention. The percentage of cells entering the G2/M phase is reduced by about 45% with the strain of the invention.

### Statistical Analyses

The significance of the aforementioned results was evaluated by an analysis of the one-factor variance (ANOVA) followed by a Tukey test for the multiple comparison of average. Regarding the experiment of the transport of calcium through a membrane of Caco-2 cells, the post-ANOVA test is a Dunn's test resulting in a comparison with the average of the control condition. The difference between the averages is considered to be significant for a p value <0.05. The statistical tests were conducted under the software R (R core team, 2016, Vienne, Austria), on the R Commander package.

### Results Relating to The Strain VFH049

The Lactobacillus helveticus VFH049 strain was deposited by the Applicant, on 19 Feb. 2019 at the CNCM (Collection Nationale de Cultures de Microorganismes), 25 rue du Docteur ROUX 75724 Paris Cedex 15 under order number CNCM-I-5403 (deposit according to the Budapest Treaty).

This strain is described in patent application FR 1902851 the content of which is incorporated by reference to the present application. It was isolated from mare's milk coming from Mongolia.

The strain VFH049 produces by fermentation of cow's milk molecules (peptides) capable of inhibiting ACE. The fermentation is conducted in the following way:

The strain is cultivated is a bioreactor of 500 mL (MiniBio 500, my-Control, Applikon Biotechnology, Delft, Netherlands). The medium is constituted of skim milk (10 g/L (see preceding paragraph), autoclaved 30 min at 110° C. The strain is inoculated at an OD₆₀₀ equal to 0.3. The fermentation is conducted for 72 h at a constant temperature of 40° C. with an agitation of 300 rpm in a final volume of 330 mL. The anaerobic condition is obtained by injection of N₂ into the bioreactor at a flow rate of 20 mL/min. All throughout the experiment, the pH is maintained at 6 thanks to HCl (1 M) and NaOH (3 M) solutions, the supply is carried out using two peristaltic pumps located on the control unit of the bioreactor. Samples of about 5 ml are taken in aseptic conditions at 0, 2, 17, 24, 41, 48, 65 and 72 h of fermentation for analysis of the growth of the bacterial strains as well as the change in the concentration in peptides. The latter is evaluated by a dosage of the reagent FC after a TCA precipitation. A volume of 1 mL of fermentate is diluted ( 1/10) in a solution of EDTA 2% (m/v) at pH 12 then centrifuged at 13,400 rpm for 10 min, and 25 μL of supernatant are analyzed by SEC. The bacterial residue is resuspended in 1 mL of PBS buffer to measure the OD₆₀₀ so as to evaluate the bacterial growth. After 72 h of fermentation, all of the fermentate is recovered then centrifuged at 10,000 rpm for 10 min, the supernatant is then stored at −20° C. until the analysis. The concentration in dry matter in the fermentates is measured in the dessicator (XM60, Precisa, Poissy, France) and is still equal to 100 g/L. So as to be able to confirm the impact of the bacterial fermentation on the properties of the final product, a control is carried out (CTLF). This is the fermentation medium incubated in the same conditions but not inoculated. After 72 h of incubation, this medium is centrifuged and stored.

In order to remove the non-hydrolyzed proteins, a portion of the crude fermentate is fractionated using an ultrafiltration membrane. The membrane is a Hydrosart® cassette (Sartocon® Slice Hydrosart® Cassette, Sartorius, Gottingen, Germany) of 0.1 m² with a cutoff threshold of 10 kDa connected to a Sartocon® Slice 200 Holder system (Sartorius, Göttingen, Germany). The supply of the fermentate is carried out by a peristaltic pump and the pressure of the retentate is maintained at about 1 bar during the filtration. Thus, the ultrafiltration permeate (UFP) containing molecules less than 10 kDa is separated from the retentate (UFR). The two sub-fractions are then stored at −20° C. until the analysis. The ultrafiltration permeates are dried to 10% of the initial volume by centrifugal evaporation (miVac, Gene Vac, Ipswich, United Kingdom) for 15 h at 40° C. The concentration in dry matter in the products (fermentate and ultrafiltered fermentate) is then measured in the dessicator (XM60, Precisa, Poissy, France).

The aforementioned filtrate which is a mixture of peptides has an AVE inhibiting activity.

### Absorption of The Calcium by The Strain VFH049

The same experiments as hereinabove were conducted with the strain VFH049.

The results show that the TEER increases during the incubation time with the strains as well as in the control condition (PBS buffer). Thus, for the strain VFH049, the TEER passes from 100 % to t=0 at 245% after 24 h quasi-linearly. Moreover, it is observed that the strain VFH049 does not make it possible to significantly modulate the absorption after 7 h of contact in relation to the PBS buffer. However, after 24 h of incubation, the concentration in calcium decreases in the basal compartment for the control condition, while a significant increase in the quantity of calcium is observed for the strain VFH049. Thus, for the strain VFH049, the ratio concentration at t=2 h/concentration at t=0 reaches the value of 1.08.

The emission fluorescence ratio on the basal emission basal is 1.08 for PBS, 1.23 for the control fermentate and 1.31 for the fermentate produced by the strain VFH049. When the ultrafiltered fermentates are tested, the effect is pronounced for the strain VFH049. The strain VFH049 thus favors the absorption of calcium.

The samples of fermentate obtained by fermentation of the strain VFH049 were put into contact with the Caco-2 cells and the variations in the expression of the gene trpv6 were then studied by qPCR.

The fermentate produced by the strain VFH049 results in an overexpression of the gene 20 times greater than the control. The effect of the ultrafiltered fermentates is less pronounced, even non-significant although a trend in the induction of the gene is clearly visible (results not shown). The strain fermentates are therefore capable of modulating both the incorporation of the calcium and the expression of trpv6 in relation to non-fermented milk.

Regarding the strain VFH049, it results in an increase in the expression of the gene coding for the transporter of the calcium (trpv6) of at least 3 times greater than the control (results not shown).

Moreover, the % of cells adhering to the Caco-2 cells is 1.48±0.41 for the strain VFH049. The strain VFH049 is much more adherent to the intestinal cells which tends to indicate that it is more likely to have a probiotic action.

1-10. (canceled)

11. A fermented food product comprising Lactobacillus plantarum VF46A deposited at the CNCM under the number CNCM I-5301, wherein the fermented food product does not comprise dzomo's milk.

12. The fermented food product of claim 11 comprising a milk.

13. The fermented food product of claim 12, wherein the milk comprises cow's milk, goat's milk, ewe's milk, or mixtures thereof.

14. The fermented food product of claim 11 comprising a plant milk.

15. The fermented food product of claim 14, wherein the plant milk comprises oat milk, Kamut wheat milk, spelt milk, millet milk, barley milk, rye milk, rice milk, almond milk, soy milk, walnut milk, coconut milk, hemp milk, hazelnut milk, peanut milk, cashew milk, pistachio milk, or mixtures thereof.

16. The fermented food product of claim 11, which is a yogurt.

17. The fermented food product of claim 11, which is a kefir.

18. The fermented food product of claim 11, which is a cheese.

19. A pharmaceutical composition comprising Lactobacillus plantarum VF46A deposited at the CNCM under the number CNCM I-5301.

20. The composition of claim 19 further comprising vitamin D.

21. The composition of claim 19, which is a unit dose comprising at least 10⁵ CFU of Lactobacillus plantarum VF46A.

22. The composition of claim 19 further comprising Lactobacillus helveticus VFH049 deposited at the CNCM under the number CNCM I-5403.

23. A method of treating a disease or disorder comprising administering to a subject in need thereof a therapeutically effective amount of the composition of claim 19, wherein the disease or disorder is linked to a deficiency in the intestinal absorption of calcium or is an abnormal physiological condition linked directly or not to a dysfunction of the vitamin D receptor, the TRPV6 transporter or a deregulation of the mechanisms of cellular signaling involving calcium, in particular apoptosis and cell proliferation.

24. The method of claim 23, wherein the disease or disorder is osteoporosis, type 2 diabetes, hypertension, cardiac pathologies linked to blood calcium, obesity or metabolic syndrome.

25. A method of treating cancer comprising administering to a subject in need thereof a therapeutically effective amount of the composition of claim 19.

26. The method of claim 25, wherein the cancer is colon cancer, prostate cancer, ovarian cancer or breast cancer.

27. A method of treating chronic inflammatory bowel disease comprising administering to a subject in need thereof a therapeutically effective amount of the composition of claim 19.


SEQUENCE LISTING (8 sequences)

SEQ ID NO: 1 - DNA, 20 bp, Artificial sequence
  recombinant primer
gccacctgctctatgccaag

SEQ ID NO: 2 - DNA, 22 bp, Artificial sequence
  recombinant primer
caggctgtcctagtcaggagat

SEQ ID NO: 3 - DNA, 23 bp, Artificial sequence
  recombinant primer
tgatgcggctcatcagtgccagc

SEQ ID NO: 4 - DNA, 22 bp, Artificial sequence
  recombinant primer
gtagaagtggcctagctcctcg

SEQ ID NO: 5 - DNA, 20 bp, Artificial sequence
  recombinant primer
tgctgactgtggacaactcg

SEQ ID NO: 6 - DNA, 20 bp, Artiificial sequence
  recombinant primer
tgcagcgagagcacaaagat

SEQ ID NO: 7 - DNA, 20 bp, Artificial sequence
  recombinant primer
agagtttgatcctggctcag

SEQ ID NO: 8 - DNA, 17 bp, Artificial sequence
  recombinant primer
attaccgcggctgctgg